(19) United States
(12) Patent Application Publication
(10) Pub. No.: US 2022/0356524 A1
(43) Pub. Date: Nov. 10, 2022

(54) GENE EXPRESSION SIGNATURE OF ENDOMETRIAL SAMPLES FROM WOMEN WITH AND WITHOUT ENDOMETRIOSIS

(71) Applicant: Endometrics Ltd., Claymont, DE (US)

(72) Inventors: Yana Borisovna GARAZHA (AZNAUROVA), Claymont, DE (US); Leila Vladimirovna ADAMYAN, Moscow (RU); Assia Arnoldovna STEPANIAN, Atlanta, GA (US); Daniil Mikhailovich NIKITIN, Walnut, CA (US); Andrew Vladimirovich GARAZHA, Walnut, CA (US); Maria Vladimirovna SUNTSOVA, Walnut, CA (US); Maksim Igorevich SOROKIN, Walnut, CA (US); Anton Aleksandrovich BUZDIN, Walnut, CA (US)

(21) Appl. No.: 17/739,526

(22) Filed: May 9, 2022

(30) Foreign Application Priority Data
May 7, 2021 (RU) 2021113171

(51) Int. Cl. C12Q 1/6883

(52) U.S. Cl. CPC C12Q 1/6883; C12Q 2600/112; C12Q 2600/158; C12Q 1/6869

(57) ABSTRACT

The present invention relates to the gene signature of the endometrium which may serve as a non-surgical diagnostic method for endometriosis. The signature includes five genes according to the claimed invention which are downregulated in endometrium and endometriotic lesions of patients with endometriosis, selected after comparison to the endometrium of women without endometriosis. This gene signature demonstrated a capacity of nearly perfect separation of all 52 analyzed tissue samples of patients with endometriosis (both their endometrial samples and endometriosis lesions) from 14 tissue samples of both living and cadaveric patients without endometriosis (AUC=0.982, Matthews correlation coefficient MCC=0.832).

The present application claims priority from and the benefit of Application RU No. 2021113171, filed May 7,2021, the entire contents of each of which are incorporated herein by reference for all purposes.

### TECHNICAL FIELD

The present invention relates to a method for identifying patients with endometriosis

### BACKGROUND OF THE INVENTION

Endometriosis is a benign gynecological disease histologically characterized by the presence of endometrial-like cells outside the uterus (Koninckx P. R. et al. Pathogenesis of endometriosis: the genetic/epigenetic theory//Fertil Steril. 2019;111(2):327-340). The prevalence of this disease reaches 10-15% in women of reproductive age, which accounts for approximately 200 million women worldwide (Macer M. L. et al. Endometriosis and infertility: a review of the pathogenesis and treatment of endometriosis-associated infertility//Obstet Gynecol Clin North Am. 2012;39:535-549).

Although endometriosis has been studied for over 150 years, there are still no effective biomarkers, and laparoscopy with subsequent histological analysis remains the gold standard for the diagnosis of this enigmatic disease (Kiesel L. et al. Diagnosis of endometriosis in the 21st century//Climacteric. 2019 Jun;22(3):296-302). This surgical intervention is performed strictly according to indications, under general anesthesia, and may be associated with intra- and postoperative complications. Endometriosis recurrence is also determined via laparoscopy (repeated), which is traumatic for patients in both the physical and emotional sense. Mentioned negative consequences are potentially preventable with the use of reliable non-invasive biomarkers that are effective at the early stages of endometriosis (Romeo A. et al. Which knots are recommended in laparoscopic surgery and how to avoid insecure knots//J Minim Invasive Gynecol. 2020;27(6):1395-1404).

Comparative investigation of genes and gene networks involved in endometriosis is important for understanding the underlying mechanisms (Aznaurova Y. B. et al. Molecular aspects of development and regulation of endometriosis//Reprod Biol Endocrinol. 2014;12(1):50). Currently, the “omics”—technologies are rapidly advancing, allowing for the study of the genome, transcriptome, metabolome, and proteome of different cells (genomics, transcriptomics, metabolomics, and proteomics, respectively) (Hasin Y. et al. Multi-omics approaches to disease//Genome Biol. 2017;18(1):83). Moreover, RNA sequencing (RNA-seq) is becoming a novel technical standard of quality in gene expression studies (Wang Z. et al. RNA-Seq: a revolutionary tool for transcriptomics//Nat Rev Genet. 2009;10(1):57-63; Zhang Y. H. et al. Identifying and analyzing different cancer subtypes using RNA-seq data of blood platelets//Oncotarget. 2017; 8(50) :87494-87511).

There are many published reports on gene expression studies in endometriosis, which differ in terms of the number of patients, the samples taken for analysis, the platform for sequencing, and the statistical methods used (Burney R.O. et al. Gene Expression Analysis of Endometrium Reveals Progesterone Resistance and Candidate Susceptibility Genes in Women with Endometriosis//Endocrinology. 2007;148(8):3814-3826; Zhang M. et al. Expression Profile Analysis of Circular RNAs in Ovarian Endometriosis by Microarray and Bioinformatics II Med Sci Monit. 2018;24:9240-9250; Allegra A. et al. The gene expression profile of cumulus cells reveals altered pathways in patients with endometriosis//J Assist Reprod Genet. 2014;31(10):1277-1285). All identified studies are based on individual gene expression analysis, which, unfortunately, has not shown adequate results in detecting endometriosis.

For example, Mettler L. et al. conducted a comparative analysis of gene expression in endometrial samples of women with and without endometriosis, as well as in samples of women of the control group (Mettler, L. et al. Comparison of cDNA microarray analysis of gene expression between eutopic endometrium and ectopic endometrium (endometriosis)//Journal of assisted reproduction and genetics, 2007;24(6):249-258) and revealed 13 differentially expressed genes in patients with endometriosis.

Irungu, S. et al. conducted a proteomic analysis of endometrial samples (Irungu S. et al. Discovery of non-invasive biomarkers for the diagnosis of endometriosis//Clin Proteom 2019;16:14), identified differential expression of biomarkers LUM, CPM, TNC, TPM2, and PAEP and analyzed the effectiveness of CA125, sICAM1, FST, VEGF, MCP1, MIF, and IL1R2 in the endometriosis detection. The authors noted that CA125 is the only biomarker that can be used alone to diagnose endometriosis, while other markers should be used in combination.

Gupta D. et al. performed a meta-analysis of 54 studies on endometrial biomarkers of endometriosis (Gupta D. et al. Endometrial biomarkers for the non-invasive diagnosis of endometriosis//Cochrane Database Syst Rev. 2016; Apr 20;4(4):CD012165). The effectiveness of 22 biomarkers in the early diagnosis of endometriosis was analyzed in those studies: angiogenesis and growth factors (PROK-1), cell-adhesion molecules (integrins α3β1, α4β1, β1, and α6), DNA-repair molecules (hTERT), endometrial and mitochondrial proteome, hormonal markers (CYP19, 17βHSD2, ER-α, ER-β), inflammatory markers (IL-1R2), myogenic markers (caldesmon, CALD-1), neural markers (PGP 9.5, VIP, CGRP, SP, NPY, NF) and tumor markers (CA-125). As a result, diverse data were obtained in different studies regarding numerous biomarkers expression in endometriosis, especially in the case of PGP 9.5 and CYP19 markers. The authors also noted that many studies did not reveal a statistically significant difference in biomarkers expression levels in endometrial samples of women with and without endometriosis.

Despite various types of research in this field, no minimally invasive diagnostic test has been created for endometriosis.

### BRIEF DESCRIPTION OF THE INVENTION

The objective of the present invention is to identify genetic biomarkers of endometriosis that can effectively diagnose endometriosis via endometrial sample analysis.

Another objective is to develop a non-surgical method for endometriosis diagnosis based on the analysis of identified genetic biomarkers.

The objective is solved by implementing a method for diagnosis of endometriosis or the likelihood of having endometriosis, which includes assessment of gene expression in an endometrial tissue sample and/or endometrial cells of the patient, where the downregulated expression of at least one of the genes among following—LMNA, KDM6B, CIC, PERI , and PPDPF—indicates the presence of endometriosis in this patient.

In some embodiments of the invention, a downregulated expression of at least two genes out of the following five ones—LMNA, KDM6B, CIC, PER1, and PPDPF—indicates the presence of endometriosis in this patient.

In some embodiments of the invention, a downregulated expression of at least three genes out of the following five ones—LMNA, KDM6B, CIC, PER1, and PPDPF—indicates the presence of endometriosis in this patient.

In some embodiments of the invention, a downregulated expression of at least four genes out of the following five ones—LMNA, KDM6B, CIC, PER1, and PPDPF—indicates the presence of endometriosis in this patient.

In some embodiments of the invention, a downregulated expression of each of the following genes—LMNA, KDM6B, CIC, PER1, and PPDPF—indicates the presence of endometriosis in this patient.

In some embodiments of the invention, an endometrial tissue sample and/or endometrial cells are obtained via an endometrial biopsy, via endometrial cell isolation from a menstrual blood sample, or via endometrial cell isolation from a peripheral blood sample.

In some embodiments of the invention, the endometrial biopsy means a brush biopsy or an aspiration (pipelle) biopsy.

In some embodiments of the invention, the expression of the LMNA, KDM6B, CIC, PER1, and PPDPF genes in a patient's endometrial sample is determined by analyzing the level of corresponding mRNAs, which is indicative of the mentioned genes expression.

In some embodiments of the invention, analysis of mRNA level is performed by total RNA sequencing (Next-generation sequencing (NGS)) or using reverse transcription-quantitative real-time polymerase chain reaction (RT-PCR).

In some other embodiments of the invention, mRNA analysis is performed using microarray hybridization, custom panels for gene expression, NanoString nCounter system, or other methods that allow the measurement of mRNA level.

In some other embodiments of the invention, the expression of the LMNA, KDM6B, CIC, PER1, and PPDPF genes in a patient's endometrial sample is determined by assessing the quantification of corresponding proteins, which is indicative of the mentioned genes' expression.

In some other embodiments of the invention, the quantification of corresponding proteins is performed using mass spectrometry, ELISA, immunohistochemistry, protein microarrays, electrochemiluminescence, or other methods that allow quantification of corresponding proteins in an endometrial tissue sample and/or endometrial cells.

In some embodiments of the invention, a downregulated expression of LMNA, KDM6B, CIC, PERI , and PPDPF genes means the gene expression level which is lower than the corresponding gene expression level in an endometrial sample of a patient without endometriosis.

The task is solved by the development of the system for the diagnosis of endometriosis in a patient, comprising hardware logic designed or configured to perform operations, including:

(a) receiving information regarding expression levels of the LMNA, KDM6B, CIC, PER1, and PPDPF genes from a biological sample of endometrial tissue taken from the specified patient,

(b) applying expression levels to a predictive model relating expression levels of mentioned genes to endometriosis; and

(c) evaluation of an output of said predictive model to assess the likelihood of endometriosis presence in a patient,

wherein the presence of endometriosis in a particular patient is indicated by a downregulated expression of at least one of the genes out of the following ones: LM NA, KDM6B, CIC, PER1, and PPDPF.

In some embodiments of the invention, as a device for obtaining information about the level of expression of the LMNA, KDM6B, CIC, PER1, and PPDPF genes in an endometrial tissue sample and/or endometrial cells, the system contains a microarray.

In some other embodiments of the invention, the system comprises a sequencer, next-generation sequencer (NGS), rtPCR system, mass spectrometer, luminometer, spectrophotometer, fluorimeter, or other devices that can be used to obtain information about the gene expression level in an endometrial tissue sample and/or endometrial cells.

When implementing the invention, the following technical results are achieved:

for the first time, gene expression signature of endometrial samples of women with endometriosis was identified, including the LMNA, KDM6B, CIC, PER1, and PPDPF genes, which allows for effectively differentiate of endometrial samples of patients with endometriosis from endometrial samples of women without this disease;

a method for non-invasive diagnosis of endometriosis was developed, which is based on the analysis of identified genetic biomarkers included in the gene expression signature of endometrial samples in endometriosis;

the performed analysis shows that the identified method demonstrates high sensitivity and specificity in terms of the diagnosis of endometriosis;

a system for diagnosing endometriosis in a patient has been developed, comprising hardware logic designed or configured to perform operations related to the analysis of identified genetic biomarkers included in the gene expression signature of endometrial samples in endometriosis.

### BRIEF DESCRIPTION OF THE FIGURES

FIG. 1. Histological images of endometrium of patient without endometriosis (A), endometrium of patient with endometriosis (B), and peritoneal lesion of the same patient (C).

FIG. 2. 2-dimentional PCA plot of all tissue samples used in this study.

FIG. 3. Scheme of preliminary gene signature construction pipeline used in this study.

FIG. 4. Scheme of five-fold cross validation used in this study.

FIG. 5. Histogram of gene occurrences in the five instances of preliminary gene signature generation procedure.

FIG. 6. Distribution of 5-gene signature score for 52 endometrial samples and endometriotic lesions of patients with endometriosis and 12 tissue samples of women without endometriosis. Threshold value is shown as vertical red line.

FIG. 7. Clustering analysis of all samples from an external dataset (GSE134056)

FIG. 8. A. Distribution of 5-gene signature score for 14 endometrial samples and endometriotic lesions of patients with endometriosis and 20 tissue samples of women without endometriosis from the dataset GSE134056.

FIG. 8. B. ROC-AUC analysis for 5-gene signature score predictive capacity to discriminate between normal endometrium and endometriosis in the dataset GSE134056. AUC =0.72.

Definitions and terms

The following terms and definitions are used in this document unless otherwise specified. References to techniques used in the description of this invention refer to well-known methods, including modifications of these methods and replacements of these methods with equivalent methods well known by specialists.

In the documents of this invention, the terms “comprise”, “comprising”, “contain”, “containing” etc. as well as “include”, “including”. etc. are interpreted as “comprises, among other things” (or “includes, among other things”). These terms are not intended to be construed as “comprising only of”. Thus, as used herein the term “comprising” means that the named elements are included, but other elements (e.g., unnamed signature genes) may be added and still represent a composition or method within the scope of the claim. The transitional phrase “consisting essentially of” means that the associated composition or method encompasses additional elements, including, for example, additional signature genes, that do not affect the basic and novel characteristics of the disclosure.

As used herein, the singular terms “a,” “an,” and “the” include the plural reference unless the context clearly indicates otherwise.

A sample of endometrial cells or endometrial tissue implies any cells and tissue structures of the endometrium, including both the stromal and glandular components. For example, secretory or ciliated cells of the epithelial component of the endometrium, and fibroblast-like cells of the stromal component of the endometrium, but not limited to, as well as any combinations thereof, can be used as a sample for analysis. An endometrial tissue sample, peripheral blood sample, and/or menstrual blood sample containing endometrial cells or tissue structures might be used as a sample for analysis. In some preferred embodiments, the sample used for analysis represents an endometrial biopsy.

A gene signature or gene expression signature is a single or combined group of genes in a cell with a unique characteristic pattern of gene expression that occurs as a result of an altered or unaltered biological process or pathogenic medical condition.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “patient” in this document refers to a human subject (woman). According to the invention, the patient may be a subject of reproductive age, but not limited to; the patient may also be a subject of pre- and post-reproductive age.

The term “sensitivity” as used herein is equal to the number of true positives divided by the sum of true positives and false negatives.

The term “specificity” as used herein is equal to the number of true negatives divided by the sum of true negatives and false positives.

The term “LMNA” or “human LMNA” (lamin A/C) refers to a gene with a sequence listed at the National Center for Biotechnology Information (NCBI) under the number NG_008692 (https://www.ncbi.nlm.nih.gov/nuccore/ NG_008692), as well as to allelic variants of this gene (isoforms) present in the genome of patients. The unique identifier for this gene according to the Human Gene Nomenclature Committee of the European Bioinformatics Institute (HGNC): 6636.

The term “KDM6B” or “human KDM6B” (lysine demethylase 6B) refers to a gene with a sequence listed at the National Center for Biotechnology Information (NCBI) under the number NG_053032 (https://www.ncbi.nlm.nih.gov/nuccore/NG_053032), as well as to allelic variants of this gene (isoforms) present in the genome of patients. The unique identifier for this gene according to the Human Gene Nomenclature Committee of the European Bioinformatics Institute (HGNC): 29012.

The term “CIC” or “human CIC” (capicua transcriptional repressor) refers to a gene with a sequence listed at the National Center for Biotechnology Information (NCBI) under the number NG_042060 (https://www.ncbi.nlm.nih.gov/nuccore/NG_042060), as well as to allelic variants of this gene (isoforms) present in the genome of patients. The unique identifier for this gene according to the Human Gene Nomenclature Committee of the European Bioinformatics Institute (HGNC): 14214.

The term “PERI” or “human PERI” (period circadian regulator 1) refers to a gene with a sequence listed at the National Center for Biotechnology Information (NCBI) under the number NC_000017 (https://www.ncbi.nlm.nih.gov/nuccore/ NC_000017.11), as well as to allelic variants of this gene (isoforms) present in the genome of patients. The unique identifier for this gene according to the Human Gene Nomenclature Committee of the European Bioinformatics Institute (HGNC): 8845.

The term “PPDPF” or “human PPDPF” (pancreatic progenitor cell differentiation and proliferation factor) refers to a gene with a sequence listed at the National Center for Biotechnology Information (NCB!) under number NC_000020 (https://www.ncbi.nlm.nih.gov/nuccore/NC_000020.11), as well as to allelic variants of this gene (isoforms) present in the genomes of patients. The unique identifier for this gene according to the Human Gene Nomenclature Committee of the European Bioinformatics Institute (HGNC): 16142.

The technical and scientific terms in this application have the standard meanings generally accepted in the scientific and technical literature unless otherwise defined.

### DETAILED DESCRIPTION OF THE INVENTION

Endometriosis is a disease where tissue similar to the lining of the uterus, the endometrium, grows outside the uterus. Endometriosis is characterized by pelvic pain, dysmenorrhea, dyspareunia, and dysuria, and in 46-50% of cases, patients are diagnosed with primary or secondary infertility. According to The Endometriosis Association, 66% of women with endometriosis report the onset of pelvic pain prior to age 20 (Ballweg M L. Big picture of endometriosis helps provide guidance on the approach to teens: comparative historical data show endo starting younger, is more severe. J Pediatr Adolesc Gynecol. 2003 Jun;16(3 Suppl):S21-6.). Since the symptoms of endometriosis are not pathognomonic for this disease, the diagnostic delay is 6-10.4 years, according to statistics in different countries (Ghai V, Jan H, Shakir F, Haines P, Kent A. Diagnostic delay for superficial and deep endometriosis in the United Kingdom. J Obstet Gynaecol. 2020 Jan;40(1):83-89.; Hudelist G, Fritzer N, Thomas A, Niehues C, Oppelt P, Haas D, Tammaa A, Salzer H. Diagnostic delay for endometriosis in Austria and Germany: causes and possible consequences. Hum Reprod. 2012 Dec;27(12):3412-6.). Such a delay in diagnosis in most cases leads to the chronicity of symptoms, an increase in the number and size of endometriosis lesions, and infertility.

Within the framework of the present invention, for the first time, a diagnostic gene expression signature of endometrium in endometriosis has been developed to differentiate the endometrium of women without endometriosis from the endometrium of women with endometriosis.

In conducted studies, the RNA sequencing of endometrial samples and endometriosis lesions of patients with endometriosis and endometrial samples of patients without this disease was performed. The two-step differential gene expression analysis of endometrial samples and endometriotic lesions of patients with endometriosis in comparison with endometrial samples of women without endometriosis was conducted. First, differentially expressed genes were extracted from a comparison between endometrial samples of the patients with and without endometriosis. Second, genes were selected to provide a preliminary gene expression signature with a maximum AUC score for separation between endometrial samples and endometriosis lesions of the patients with endometriosis and endometrial samples of the patients without endometriosis. A subsequent 5-fold cross-validation procedure showed that the preliminary gene signatures had high predictive power (AUC scores 0.95-1) and shared a high number of common genes (5 genes) which enabled the construction of a final gene signature. Based on this analysis, a characteristic signature of five genes downregulated in the endometrial samples and endometriotic lesions of patients with endometriosis in comparison with the endometrial samples of women without endometriosis (LMNA, KDM6B, CIC, PER1, and PPDPF) was generated.

The identified gene expression signature allowed effective separation of all samples obtained from patients with endometriosis (endometrial samples and endometriosis lesions) from samples obtained from the control group of women without endometriosis and demonstrated a significant predictive power (area under the curve=0.982, Matthews correlation coefficient=0.832). The method of 5-fold cross-validation of the proposed two-step procedure confirmed the ability to generate conservative and robust gene signatures having significant predictive power (AUC>0.85) using real-world clinical data. Cross-validation also made it possible to avoid the so-called “type III error”. Additional validation of gene expression signature on a group of other tissue samples of the control group (endocervix, ovarian epithelium) made it possible to verify the specificity of the identified gene signature for endometrial tissue.

Thus, as a result of the studies, it was found that the LMNA, KDM6B, CIC, PER1, and PPDPF genes are effective biomarkers of endometriosis. More specifically, a decreased expression level of one or more of these genes in the endometrial tissue samples or endometrial cells of a patient (compared to these genes' expression level in the endometrial sample or endometrial cells of a patient without endometriosis) is indicative of the presence of endometriosis in that patient.

In some cases, downregulated expression of only one gene out of the following five—LMNA, KDM6B, CIC, PER1, and PPDPF genes—in endometrial tissue samples and/or endometrial cells might be sufficient for diagnosis of endometriosis; for example, downregulated expression of the LMNA gene alone may indicate the presence of endometriosis; or the presence of endometriosis may be indicated by downregulated expression of the KDM6B gene only, or the presence of endometriosis may be indicated by downregulated expression of the CIC gene alone, or the presence of endometriosis may be indicated by downregulated expression of the PER1 gene alone, or the presence of endometriosis may be indicated by downregulated expression of the PPDPF gene alone.

In more preferred embodiments, i.e., of high likelihood, the presence of endometriosis in the patient is indicated by a downregulated expression of at least two genes out of the following five—LMNA, KDM6B, CIC, PER1, and PPDPF. In more preferred embodiments, the presence of endometriosis in the patient is indicated by a downregulated expression of at least three genes selected out of the following five—LMNA, KDM6B, CIC, PER1, and PPDPF. Decreased expression levels of four or all five genes—LMNA, KDM6B, CIC, PER1, and PPDPF genes—are even more likely to indicate the presence of endometriosis in the patient.

Thus, based on the conducted studies and the obtained data, an in vitro method for endometriosis diagnosis was developed, including an assessment of gene expression level in an endometrial tissue sample and/or endometrial cells of the patient, where a reduced level of expression of at least one gene out of the following ones—LMNA, KDM6B, CIC, PER1, and PPDPF—indicates the presence of endometriosis in this patient.

In some embodiments of the invention during the diagnosis the average expression level of the genes LMNA, KDM6B, CIC, PER1, and PPDPF can be calculated for the patient's sample. To do this, the arithmetic mean of the logarithm of the normalized number of reads uniquely mapping to the LMNA, KDM6B, CIC, PER1, and PPDPF genes in the patient is calculated and compared with the average level of expression of these genes in the norm (i.e., in subjects without endometriosis). The value of the average level of expression below the level defined as “normal” (i.e., measured in subjects without endometriosis), indicates the presence of endometriosis in the patient.

Diagnosis using mean expression level can also be performed on four genes selected from LMNA, KDM6B, CIC, PER1, and PPDPF. For this, the level of expression of any four genes selected from LMNA, KDM6B, CIC, PER1, and PPDPF is measured in the diagnosed patient. Then the obtained average value is compared with the average level of expression of the same four genes in the norm (i.e., in subjects without endometriosis). The value of the average expression level below the level defined as “normal” (i.e., measured in subjects without endometriosis), indicates the presence of endometriosis in the patient.

The proposed in vitro method for endometriosis diagnosis may additionally include the assessment of other biomarkers levels and/or other measures aimed at endometriosis diagnosis in a patient. For example, one or more additional biomarkers may be selected from differential diagnostic biomarkers, predictive biomarkers, biomarkers suitable for detecting endometriosis, and biomarkers for classifying endometriosis.

In some embodiments of the invention, the patient may have symptoms characteristic of endometriosis (eg, pelvic pain, irregular menstrual cycle, heavy menstrual bleeding, infertility, etc.). In some other embodiments, the patient may be asymptomatic, and the invention may be used to rule out asymptomatic endometriosis, for the differential diagnosis from other diseases, etc. In particular, the proposed diagnostic method can be used for the following purposes:

early diagnosis of endometriosis in patients of reproductive age (15-49 years old) with pelvic pain, dyspareunia, infertility;

differential diagnosis in patients with dysuria and dyschezia;

endometriosis screening in adolescents;

monitoring the effectiveness of endometriosis treatment (both hormonal and surgical).

According to the invention, a sample of endometrial tissue and/or endometrial cells for endometriosis diagnosis can be obtained, for example, via endometrial biopsy (brush biopsy or aspiration biopsy), via isolation of endometrial cells from a menstrual blood sample, or via isolation of endometrial cells from a peripheral blood sample, but not limited to these methods. If necessary, the sample can be subjected to various well-known methods of preparation and storage after collection (for example, fixation, storage, freezing, lysis, homogenization, DNA or RNA extraction, ultrafiltration, concentration, etc.). The sample may also be processed for further analysis. For example, the sample can be processed for cell lysis using known buffers for lysis, sonication, electroporation, etc., with purification and amplification occurring as needed, which is clear to the experts in this field. In addition, the reactions can be carried out in a variety of ways, which is clear to the experts in this field.

The expression level of a signature gene (LMNA, KDM6B, CIC, PER1, and PPDPF) can be assessed by quantifying the amount (the absolute amount or concentration) of the signature gene product, such as the protein and RNA transcript encoded by the signature gene, and the protein and RNA transcript fragments in the sample. Currently, various methods for determining the level of gene expression in biological samples of cells and tissues are well developed, all of which can be used within the framework of the present invention; the accuracy, simplicity, and cost of measuring the expression level of genes selected from LMNA, KDM6B, CIC, PER1, and PPDPF may be decisive in choosing one or another method, but this is not a limitation of the present invention. In some preferred embodiments, the expression of each of the LMNA, KDM6B, CIC, PER1, and PPDPF genes are analyzed. However, in some other embodiments of the invention, for example, in order to reduce the time and cost of analysis, or, for example, on a specific population, the expression level of 1-4 genes selected from LMNA, KDM6B, CIC, PER1, and PPDPF can be evaluated.

Quantification of the LMNA, KDM6B, CIC, PER1, and PPDPF gene expression levels can be carried out by measuring the level of the corresponding mRNA or protein. Target mRNA analysis can be performed using methods such as total RNA sequencing (NGS), quantitative real-time reverse transcription PCR (rtPCR), comparative genomic hybridization on the array (CGH, microarray hybridization), high-throughput parallel RNA sequencing (RNA-Seq), Nanostring direct digital detection technology (Nanostring nCounter), but not limited to. The analysis of the target protein can be carried out using mass spectrometry, enzyme immunoassay (ELISA), Nanostring direct digital detection technology (Nanostring nCounter), etc. Other methods can also be used to quantify the level of gene expression, such as measuring the gene methylation level that can be correlated with levels of expression or a measure of the level or activity of the protein products of the genes. The level of methylation can be determined using methods known in the art (see, for example, US6200756).

Thus, a marker for determining the genes' expression levels according to the invention can be:

LMNA mRNA, cDNA or complement;

KDM6B mRNA, cDNA or complement;

CIC mRNA, cDNA or complement;

PERI mRNA, cDNA or complement;

PPDPF mRNA, cDNA or complement.

When using quantitative reverse transcription real-time PCR (rtPCR) to analyze the signature genes expression level, primers to amplify LMNA, KDM6B, CIC, PER1, and/or PPDPF sequences can be created using primer design software such as, for example, Oligo Calc and/or Primer 3, well known to experts in this field (Chemeris D. A. et al. Design of primers for polymerase chain reaction (brief review of software and databases)//Biomics, 2016, 8, N3, 215-238). Probes for detecting LMNA, KDM6B, CIC, PER1, and/or PPDPF can be obtained from any of numerous sources depending on the desired application (e.g., using the primers described above and suitable reagents).

In some particular embodiments of the invention, the gene signature can be evaluated based on quantitative calculations.

The threshold value for a particular device, which is used to measure the level of gene expression, should be determined. This threshold should be established by calculating and minimizing the false positive rate (Type I error) and the false-negative rate (Type II error) of the endometriosis diagnosis in patients with a predetermined status of endometriosis (i.e., the status of its presence or absence).

Next, the expression levels of the LMNA, KDM6B, CIC, PER1, and PPDPF genes are measured, and the value of the gene expression signature is calculated, and this value is compared with the threshold value determined for this device to diagnose endometriosis in a patient. The value of the gene expression signature above the threshold indicates the presence of endometriosis in the patient. On the contrary, the value of the gene expression signature below the threshold value indicates a low probability of the presence of endometriosis in the patient.

For example, when using the total RNA sequencing method to measure the gene expression level, the gene signature value of the LMNA, KDM6B, CIC, PER1, and PPDPF genes is calculated as the sum of the logarithms of the normalized gene expression according to the formula:

log₁₀(LMNA expression)—log₁₀(KDM6B expression)—log₁₀(CIC expression)—log₁₀(PER1 expression)—log₁₀(PPDPF expression).

Each gene expression is then measured in normalized read counts, which are uniquely mapped to that gene after the analysis of RNA sequencing data.

Calculations showed that when using the Illumina HiSeq 3000 device for total RNA sequencing, the optimal diagnostic threshold value is “-14”.

In clinical practice, the significance of Type I and Type II errors may be different, and therefore the threshold value can be changed accordingly.

The invention also relates to a system for endometriosis diagnosis in a patient, which includes: a device configured to determine the expression levels of the LMNA, KDM6B, CIC, PER1, and/or PPDPF genes in a biological sample of endometrial tissue and/or endometrial cells; and hardware logic designed or configured to perform operations to apply expression levels to a predictive model linking expression levels of mentioned genes to endometriosis; and evaluating an output of said predictive model to assess the probability of endometriosis presence in the patient; wherein the presence of endometriosis in a particular patient is indicated by a reduced level of expression of at least one gene out of the following five—LMNA, KDM6B, CIC, PER1, and PPDPF.

A device configured to determine expression levels of signature genes in a biological sample of endometrial tissue and/or endometrial cells may vary depending on the methodology used to determine the gene expression level, as well as the specific configuration of the device. Examples of devices used for gene expression level assessment in a biological sample are clear to the experts in this field.

The analysis of expression levels of signature genes and the formulation of diagnosis based on it is usually performed using various computer executed algorithms and programs.. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel.

The present invention also relates to compositions and kits for endometriosis diagnosis in a subject. A kit is any product (e.g., a package or container) comprising at least one reagent, e.g., a probe, for detecting the expression of a signature gene according to the present invention. The kits and compositions of the invention may contain reagents necessary to analyze the level of expression of at least one gene out of the following ones—LMNA, KDM6B, CIC, PER1, and PPDPF. Additionally, the kits may also contain the necessary reagents for the preparation of a biological sample for analysis, storage, etc.

In one of the particular embodiments of the invention, the diagnosis of endometriosis can be performed as follows:

1) Collection of endometrial cells from the patient (via endometrial biopsy, isolation of endometrial cells from a peripheral blood sample, or isolation of endometrial cells from a menstrual blood sample).

2) Placement of the endometrial sample in a tube containing RNA Later solution for RNA stabilization.

3) Isolation of RNA just prior to libraries preparation for sequencing using Direct-zol™ RNA MiniPrep (Zymo Research) with TRI Reagent (MRC) according to the manufacturer's instructions.

4) Measurement of RIN (RNA Integrity Number) using an Agilent 2100 bioanalyzer. Measurement of RNA concentration using Agilent RNA 6000 Nano or Qubit RNA Assay Kits.

5) Ribosomal RNA depletion and library preparation for sequencing using the KAPA RNA Hyper c RiboErase (KAPA Biosystem) Kit. Assessment of library concentration and its quality using Qubit ds DNA HS Assay kit (Life Technologies) and Agilent Tapestation (Agilent).

6) RNA sequencing using Illumina HiSeq 3000 equipment (Illumina, Inc., San Diego, CA) for single-end sequencing, 50 bp read length, for approximately 30 million raw reads per sample. The data quality check using the Illumina SAV.

7) Bioinformatic analysis of the RNA sequencing data using the endornetriosis gene expression signature.

8) Preparation of a report regarding performed analysis, describing the expression level of the genes included in the gene expression signature of the endometrium in endometriosis and indicating the likelihood of endometriosis presence in a particular patient.

The following examples are given for the purpose of disclosing the characteristics of the present invention and should not be construed as limiting the scope of the invention in any way.

In the following example, the studies aimed at identifying and validating the gene expression signature of endometrium in patients with endometriosis by the invention have been described in detail.

Study Design

After institutional review board approval and obtaining the inform written consents, the tissue samples were collected from 19 patients with endometriosis (endometrial and endometriotic samples) and 6 asymptomatic patients with incompetent uterine scar after cesarean section and confirmed absence of endometriosis and adenomyosis (endometrial samples) who underwent laparoscopic surgery.

### Inclusion and Exclusion Criteria

Endometriosis group. The inclusion criteria for this group were: reproductive age (18-45 years), both surgically and histologically verified diagnosis of stage III or IV endometriosis, regular menstrual cycle, late proliferative/early secretory menstrual cycle (days 8 to 21), absence of any chronic pathology (including diabetes mellitus, chronic renal disease, cardiovascular disease, and inflammatory diseases). The disease stage was determined according to the revised American Society for Reproductive Medicine classification. Endometrioid ovarian cysts and infiltrative endometriosis were identified before surgery in all cases by transvaginal ultrasound. These and peritoneal endometriotic lesions were seen laparoscopically. Patients in the endometriosis group underwent laparoscopic endometriosis excision and endometrial biopsy with histological confirmation of the diagnosis of endometriosis and exclusion of any endometrial pathology.

Control non-endometriosis group. The inclusion criteria for this group were: reproductive age (18-45 years), diagnosis of uterine scar incompetence after cesarean section, no laparoscopic evidence of endometriosis, hysteroscopically and histologically verified absence of any endometrial pathology, and histologically proven absence of adenomyosis and inflammation in uterine scar. In total, 20 women underwent laparoscopic excision of incompetent uterine scar after cesarean section with hysteroscopical control and endometrial biopsy, among whom in 6 women the detailed histopathologic investigation showed no endometriosis or adenomyosis of the uterine scar.

Control/verification cadaveric non-endometriosis group. The inclusion criteria for this group were: cadaveric patients of reproductive age (18-45 years) who had consented for organ donation for research purposes, as well as histologically confirmed absence of endometriosis and any other gynecological pathology. We included cadaveric patients due to the ethical aspects of obtaining tissue samples from healthy women without endometriosis with no indications for surgery. This control/verification group included previously RNA-seq profiled tissue samples (endocervix, ovarian surface epithelium) of 8 randomly selected healthy female cadaveric patients of 32-38 years of age. Endometrium samples of cadaveric patients were not included in the study as one of the objectives was to verify identified gene expression signature using other tissue samples of healthy donors.

Exclusion criteria for all patients were: hormone therapy and IUD use for over 1-year preceding surgery; other diseases of the uterus, fallopian tubes, and ovaries, based on clinical presentation of all patients studied, based on the imaging and lab work for all patients who underwent operations.

### Sample Collection

The experimental control group contained four ovarian surface epithelium samples of cadaveric patients without endometriosis, four endocervical tissue samples of cadaveric patients without endometriosis, and four endometrial samples from living women without endometriosis. All tissue samples from cadaveric donors without endometriosis were previously RNA-seq profiled using the same reagents and protocols (Suntsova M. et al. Atlas of RNA sequencing profiles for normal human tissues//Sci Data. 2019;6(1):36).

The biopsies of peritoneal endometriotic foci after excision using cold scissors, capsules of endometrioid cysts after cystectomy, and endometrium after diagnostic curettage of the uterine cavity were placed in separate sterile tubes and immediately stabilized in RNAlater (Qiagen, Germany) and then stored at −70 ° C. The tissue samples were divided into two fragments. The first fragment of each tissue sample was analyzed histologically, and the second one was selected for sequencing.

### Studies Conducted

6 endometrial samples of women without endometriosis were compared with 15 endometrial samples of patients with endometriosis in order to identify the differentially expressed genes.
Different numbers of top differentially expressed genes from previous comparison were tested to predict the separation between the same 6 endometrial samples of women without endometriosis and 27 endometriotic samples of patients with endometriosis. This step allowed us to create preliminary gene expression signatures.
Validation of preliminary gene expression signatures. All patients were divided into 2 large groups:
all samples of patients with endometriosis (endometrial samples and endometriotic lesions)
all samples of patients without endometriosis (both living and cadaveric). These groups of samples were randomly divided into smaller parts to allow us to proceed with five-fold cross-validation of revealed preliminary gene signatures.
We analyzed similarities between preliminary gene expression signatures, for each gene we calculated its occurrence in the five preliminary signatures. We found five genes that were shared by all five preliminary signatures.
Validation of final gene expression signature. At this step, all samples were again divided into 2 groups:

all samples of patients with endometriosis (endometrial samples+endometriotic lesions)

all samples of patients without endometriosis (both living and cadaveric). For the final gene expression signature, we calculated its score for every sample in both groups. We also determined the AUC score for separation between samples in group 1 and samples in group 2, which indicates perfect separation of samples of patients with endometriosis (both endometrial and endometriotic) from samples of living and cadaveric patients without endometriosis (endometrial samples of living patients without endometriosis and tissue samples of cadaveric patients without endometriosis).

### Tissue-Based Procedures

RNA Extraction and Library Preparation

RNA extraction was performed immediately before the preparation of sequencing libraries using Direct-zol™ RNA MiniPrep (Zymo Research) with TRI Reagent (MRC), following the manufacturer's protocol. The RNA Integrity Number (RIN) was measured using Agilent 2100 bioanalyzer. Agilent RNA 6000 Nano or Qubit RNA Assay Kits were used to measure the RNA concentration. A KAPA RNA Hyper with RiboErase (KAPA Biosystem) Kit was used for further depletion of ribosomal RNA and library preparation. Different adaptors were used for multiplexing samples in one sequencing run. Library concentrations and quality were measured using a Qubit ds DNA HS Assay kit (Life Technologies) and Agilent Tapestation (Agilent). RNA sequencing was performed using Illumina HiSeq 3000 equipment for single end sequencing, 50 bp read length, for approximately 30 million raw reads per sample at UCLA Technology Center for Genomics & Bioinformatics, Department of Pathology & Laboratory Medicine, Los Angeles, Calif., USA. The data quality check was conducted using Illumina SAV. De-multiplexing was performed using Illumina Bc12fastq2 v 2.17 software.

Processing and Normalization of RNA Sequencing Data

RNA sequencing FASTQ files were processed with STAR aligner30 in ‘GeneCounts’ mode with Ensembl human transcriptome annotation (Build version GRCh38 and transcript annotation GRCh38.89). Ensembl gene IDs were converted to HGNC gene symbols using Complete HGNC dataset (https://genenames.org, database version of Jul. 13, 2017). In total, read counts values were established for 37,233 annotated genes with corresponding HGNC identifiers. All samples analyzed in this study had more than 3.5 million total number of uniquely aligned reads, which is an internal quality control threshold established according to a previous study on the subject (Suntsova M. et al. Atlas of RNA sequencing profiles for normal human tissues//Sci Data. 2019;6(1):36). Read counts were normalized using a DESeq2 package.

### Statistical Analysis

All statistical analyses were conducted using R programming language (version 3.6.2). Read counts were normalized using a DESeq2 package (function estimateSizeFactorsForMatrix). Differential expression analysis was conducted also using a DESeq2 package (function deseq). Histograms were plotted using a ggplot2 package, and schemes of signature construction were built in Excel. Principal component analysis was conducted using built-in R function prcomp. Area Under Curve (AUC) scores were calculated using ROCR package in R environment.

### Results

Biosampling

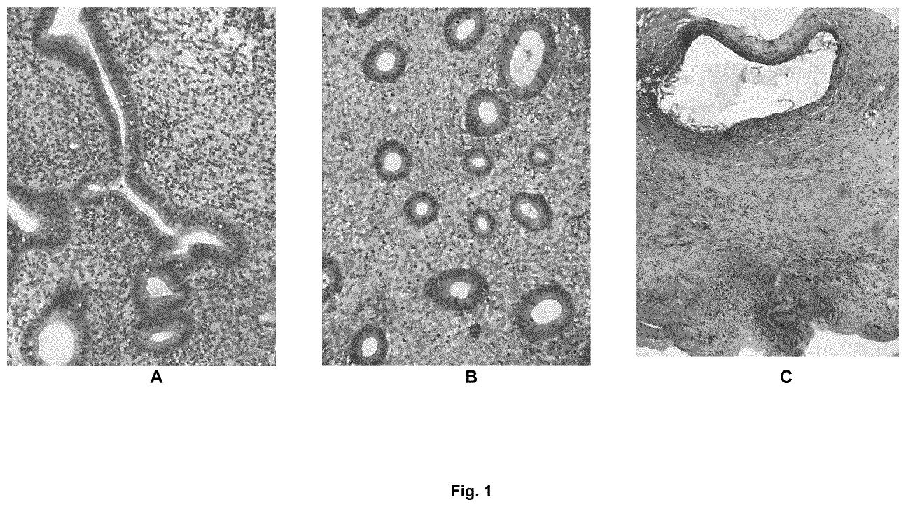

In this study, we analyzed 19 endometrial samples from patients with endometriosis, 33 endometriotic lesions from the same patients with endometriosis, and 6 endometrial samples of women without endometriosis with confirmed absence of adenomyosis and other potentially genetically determined gynecologic conditions. An additional control/verification group included various previously RNA-seq profiled tissue samples (endocervix, ovarian surface epithelium) of randomly selected healthy female cadaveric patients. The information on the biosamples is summarized in Table 1. Prior to RNA sequencing, every biosample was investigated by a pathologist (FIG. 1 A, B, C).

TABLE 1
Source and number of samples for tissues used in the analysis.

| | |
|---|---|
| | Number |
| Sample source | of samples |

Control samples (14 samples)

| | |
|---|---|
| Endometrial sample from living | 6 |
| patients without endometriosis | |
| Endocervix (simple columnar | 4 |
| epithelium) from cadaveric | |
| patients without endometriosis | |
| Ovary, outer surface (simple cuboidal | 4 |
| epithelium) from cadaveric | |
| patients without endometriosis | |

Endometrial samples of patients with endometriosis (19 samples)

| | |
|---|---|
| Endometrial sample of | 19 |
| patient with endometriosis | |

Endometriotic lesions of patients with endometriosis (33 samples)

| | |
|---|---|
| right ovarian endometriotic cyst, capsule | 12 |
| left ovarian endometriotic cyst, capsule | 6 |
| endometriosis of internal inguinal ring | 1 |
| peritoneal endometriosis | 7 |
| endometriosis of uterovesical fold | 3 |
| retrocervical endometriosis | 3 |
| abdominal wall endometriosis | 1 |

Gene Expression Data

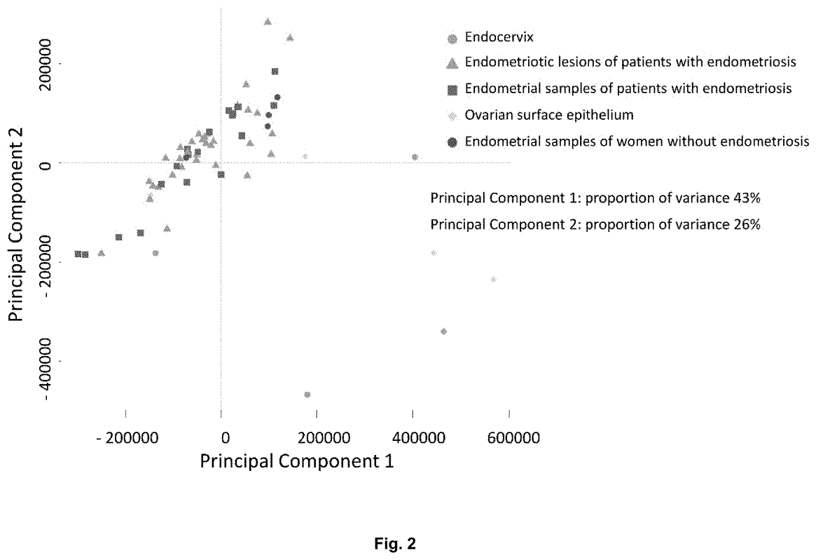

The RNA-sequencing profiles were obtained for all biosamples used in this study. Principal component analysis (PCA) assay showed that endometrial samples and endometriotic lesions of the same patients with endometriosis formed a single cluster with endometrial samples of women without endometriosis, thus confirming compatibility of the molecular data obtained (FIG. 2). The molecular data that passed quality control were then used to generate endometriosis-specific gene expression signature.

Preliminary Gene Signature Construction We performed differential expression analysis for the comparison of 6 endometrial samples of women without endometriosis versus 15 endometrial samples of patients with endometriosis. The top 500 differentially expressed genes were selected using sorting by FDR adjusted p-value, calculated with a DESeq2 package. From these 500 DEGs (Differentially Expressed Genes), different numbers of top genes were taken iteratively, and for each number n the preliminary gene signature score M(n) was calculated using the following formula:

M(n)=Σ_{Upregulated genes} log (expression)−Σ_{Downregulated genes} log(expression),

where Upregulated genes are selected differential genes with upregulated expression in endometriosis, Downregulated genes are genes with downregulated expression in endometriosis, expression means DESeq normalized expression value of a given gene, n-number of top genes sorted by FDR adjusted p-value, log is log₁₀.

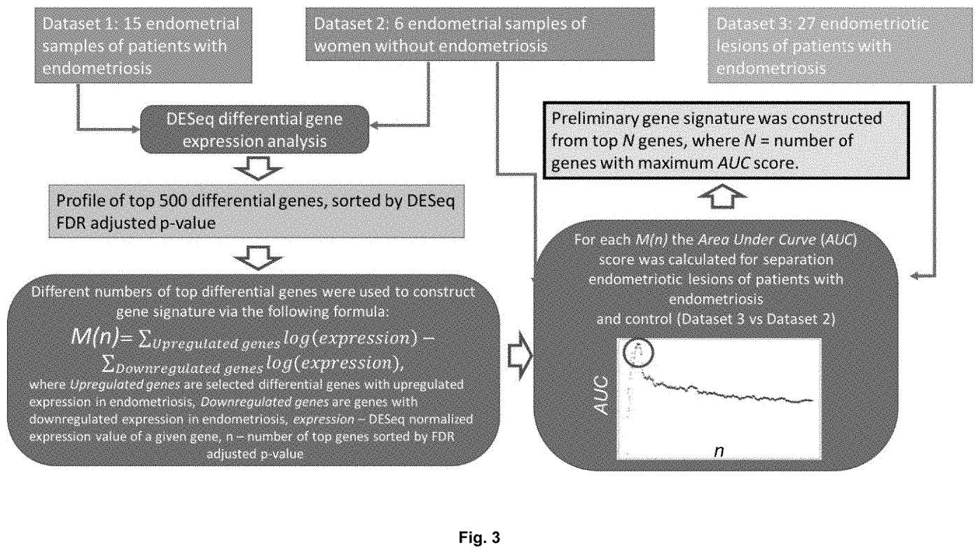

Then for each number of top differential genes n, the gene signature score M(n) was tested to predict the difference between 6 tissue samples of women without endometriosis (the same samples previously used to identify 500 differential genes) and 27 endometriotic samples of patients with endometriosis. For each n the Area Under Curve score (AUC) was calculated and n with maximum AUC score was selected to construct preliminary gene signatures (FIG. 3). The AUC value is the universal characteristic of biomarker robustness. AUC depends on the sensitivity and specificity of a biomarker. It correlates positively with the biomarker quality and may vary in an interval from 0.5 to 1. The AUC threshold for discriminating reliable and non-reliable biomarkers is typically 0.7 or 0.75. The entries having greater AUC score are considered good-quality biomarkers and vice-versa (Petrov I. et al. Molecular pathway activation features of pediatric acute myeloid leukemia (AML) and acute lymphoblast leukemia (ALL) cells//Aging (Albany N.Y.). 2016;8(11):2936-2947; Borisov N. M. et al. Signaling pathways activation profiles make better markers of cancer than expression of individual genes//Oncotarget. 2014;5(20):10198-10205).

### Cross Validation of Preliminary Gene Signatures

We used a standard five-fold cross validation procedure. Datasets of all samples of patients with endometriosis were divided into five approximately equal parts (19 endometrial samples were randomly divided into parts of 4-5 samples, whereas 33 endometriotic lesions were divided into parts of 6-7 samples). For each repeat of the five-fold cross validation, four out of five parts of endometrial samples and endometriotic lesions were taken and used to produce an optimal gene signature. Among the total of 14 tissue samples of both living and cadaveric patients without endometriosis, 6 were taken to produce a preliminary gene signature in each instance of five-fold cross validation. Selection of 6 samples was done randomly for each iteration of cross validation. Consequently, in each event of the cross validation, ⅘ of all tissue samples of patients with endometriosis and ½ of tissue samples of both living and cadaveric patients without endometriosis were used to produce a preliminary gene signature.

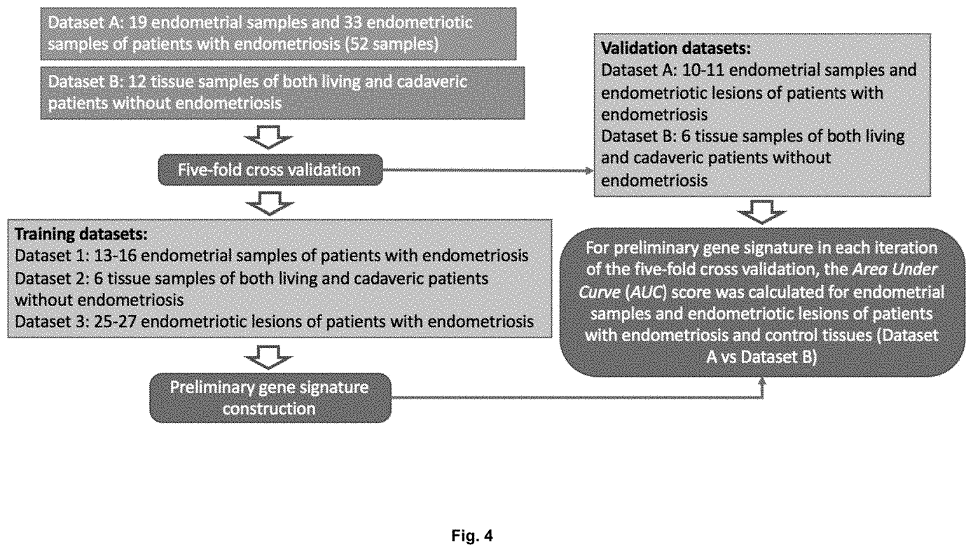

The remaining ⅕ of all tissue samples of patients with endometriosis (10-11 samples) and ½ of tissue samples of both living and cadaveric patients without endometriosis were used for a validation step in each iteration. The AUC score for the separation of all tissue samples of patients with endometriosis in the study versus tissue samples of patients without endometriosis was calculated for each preliminary gene signature (FIG. 4 and Table 2).

TABLE 2
Preliminary gene signatures and final gene signatures

| | | |
|---|---|---|
| Repeat number | Upregulated genes | Downregulated genes |

Preliminary gene signatures

| | |
|---|---|
| | LMNA, KDM6B, |
| | CIC, PER1, |
| | PPDPF, RPS11, NCOR2 |
| | LMNA, KDM6B, |
| | CIC, PER1, |
| | PPDPF, RPS11, NCOR2, |
| | LY6D, RACK1 |
| | LMNA, KDM6B, |
| | CIC, PER1, PPDPF |
| ZC2HC1A, EGLN1, | LMNA, KDM6B, |
| PHC3, KRIT1, | CIC, PER1, |
| COMMD8 | PPDPF, RPS11, NCOR2, |
| | LY6D, RACK1, |
| | EMP1, MYADM, |
| | SERPINE1, TRIM29 |
| ZC2HC1A, EGLN1, | LMNA, KDM6B, |
| PHC3, KRIT1, | CIC, PER1, |
| COMMD8 | PPDPF, RPS11, NCOR2, |
| | LY6D, RACK1, |
| | EMP1, MYADM, |
| | SERPINE1, TRIM29 |

Final gene signature

LMNA, KDM6B,
CIC, PER1, PPDPF

Cross validation AUC scores were in the range 0.95-1, whereas numbers of genes per preliminary signature in each repeat varied between 5-18 (Table 3).

TABLE 3
Results of five-fold cross validation of
preliminary gene expression signatures.

| | Number of | Number of | Total | |
|---|---|---|---|---|
| Repeat | Upregulated | Downregulated | number | Validation |
| number | genes | genes | of genes | AUC score |
| 1 | 0 | 7 | 7 | 0.97 |
| 2 | 0 | 9 | 9 | 1 |
| 3 | 0 | 5 | 5 | 1 |
| 4 | 5 | 13 | 18 | 0.98 |
| 5 | 5 | 13 | 18 | 0.95 |

### Construction of the Final Gene Signature

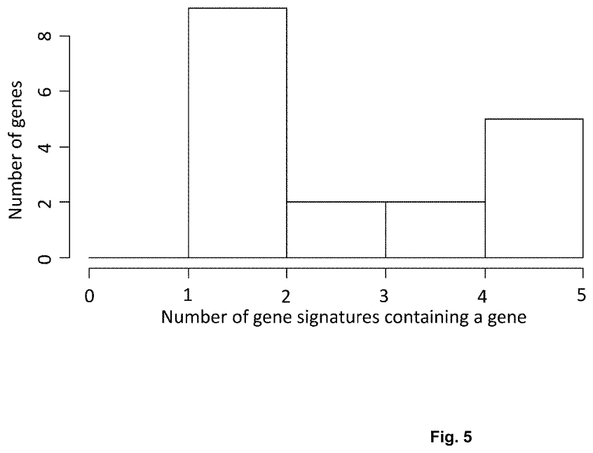

We analyzed similarities between the five gene expression signatures obtained in the study. For each gene we calculated its occurrence in the five preliminary signatures (FIG. 5). We found five genes that were shared by all five preliminary signatures in a “downregulated” state. In order to check whether this number of common genes in preliminary signatures is statistically significant, for each instance of the preliminary gene signature generating procedure, we made a random sample of a size equal to the corresponding preliminary gene signature from the total number of 37,233 annotated human genes. We intersected these five random gene samples and counted numbers of common genes in 1,000 random intersections. We found that none of these random intersections contained at least one common gene. We, therefore, conclude that q-value for significance of the 5-gene intersection experimentally observed is less than 0.001. The five genes commonly shared by all preliminary signatures are briefly characterized in Table 4. A final gene signature was then constructed with these five core genes.

TABLE 4
Gene intersection of five preliminary gene expression signatures.

| | Upregulated | | | |
|---|---|---|---|---|
| | or | | | Gene sequences |
| Gene | Downregulated | Gene function | Reference | in FASTA format |
| LMNA | Downregulated | Lamin A gene, | https://www.ncbi.nlm. | https://www.ncbi.nlm.nih.gov/ |
| | | component of | nih.gov/gene/4000 | nuccore/NG_008692.2?from= |
| | | nuclear lamina | | 4974&to=62517&report=fasta |
| KDM6B | Downregulated | Lysine-specific | https://www.ncbi.nlm. | https://www.ncbi.nlm.nih.gov/ |
| | | demethylase that | nih.gov/gene/23135 | nuccore/NG_053032.1?from= |
| | | specifically | | 10718&to=25597&report=fasta |
| | | demethylates di- or | | |
| | | trimethylated lysine | | |
| | | 27 of histone H3 | | |
| CIC | Downregulated | Transcriptional | https://www.ncbi.nlm. | https://www.ncbi.nlm.nih.gov/ |
| | | repressor, member | nih.gov/gene/23152 | nuccore/NG_042060.1?from= |
| | | of HMG group | | 5001&to=32260&report=fasta |
| PER1 | Downregulated | Expresses in | https://www.ncbi.nlm. | https://www.ncbi.nlm.nih.gov/ |
| | | a circadian | nih.gov/gene/5187 | nuccore/NC_000017.11?from= |
| | | pattern in the | | 8140472&to=815240& |
| | | suprachiasmatic | | report=fasta&strand=true |
| | | nucleus and | | |
| | | functions in | | |
| | | circadian rhythms | | |
| PPDPF | Downregulated | Pancreatic | https://www.ncbi.nlm. | https://www.ncbi.nlm.nih.gov/ |
| | | progenitor cell | nih.gov/gene/79144 | nuccore/NC_000020.11?from= |
| | | differentiation and | | 6352074&to=63522206& |
| | | proliferation factor | | report=fasta |

### Validation of Final Gene Signature

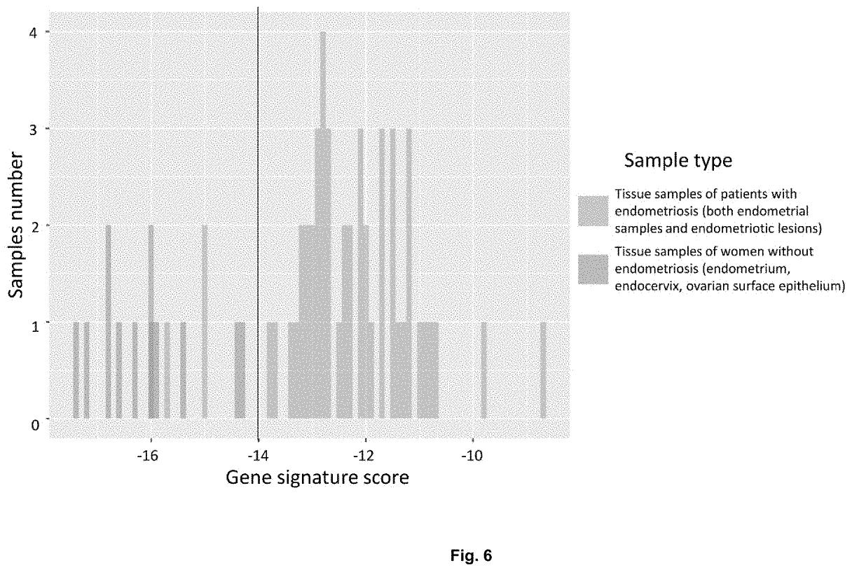

To validate the final gene signature, we took all experimental gene expression data for endometrial samples and endometriotic lesions of patients with endometriosis (52 samples) and for all tissue samples of both living and cadaveric patients without endometriosis. For the final 5-gene signature, we calculated its score for every sample, and we also determined the AUC score for separation between all studied tissue samples of women with endometriosis (endometrial and endometriotic samples) and all tissue samples of both living and cadaveric patients without endometriosis. The AUC score found for class separation was 0.982, which indicates nearly perfect separation. The corresponding final gene signature score distribution between the two classes is shown in FIG. 6.

We empirically found a threshold score value of −14 for the class discrimination by the 5-gene signature. The samples with gene signature score higher than −14 were classified as endometrial samples or endometriotic lesions of patients with endometriosis. Similarly, all samples with scores lower than —14 were predicted to be tissue samples of women without endometriosis. The error rate table for this classification is shown in Table 5.

TABLE 5
Error table for separation between
tissue samples of women without
endometriosis and all samples of
patients with endometriosis using
the final 5-gene signature with
the score threshold −14.

| | Sample is | Sample is |
|---|---|---|
| | predicted | predicted |
| | as one of | as one of |
| | women | women |
| | without | with |
| | endometriosis | endometriosis |
| Tissue samples of | 14 | 0 |
| women without | | |
| endometriosis | | |
| Tissue samples of | 4 | 48 |
| patients with | | |
| endometriosis | | |

Type 1 error rate was 0, type 2 error rate was 0.077, AUC 0.82, and Matthews correlation coefficient 0.832. The exact value of this classification threshold was established assuming identical type 1 and type 2 error rates. In clinical practice, the significances of type 1 and type 2 errors can be different, and the threshold can be, therefore, adjusted accordingly.

### Validation of Final Gene Signature on an External Dataset

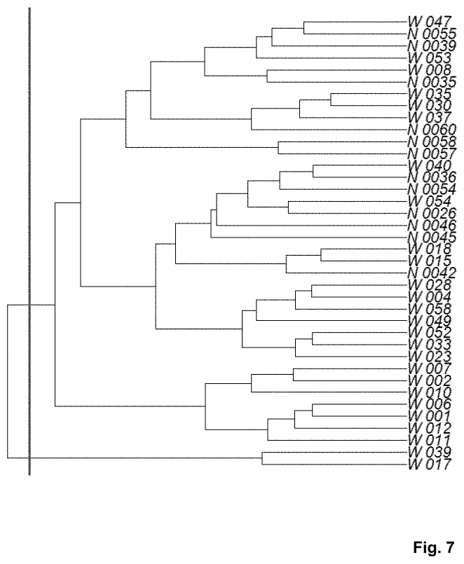

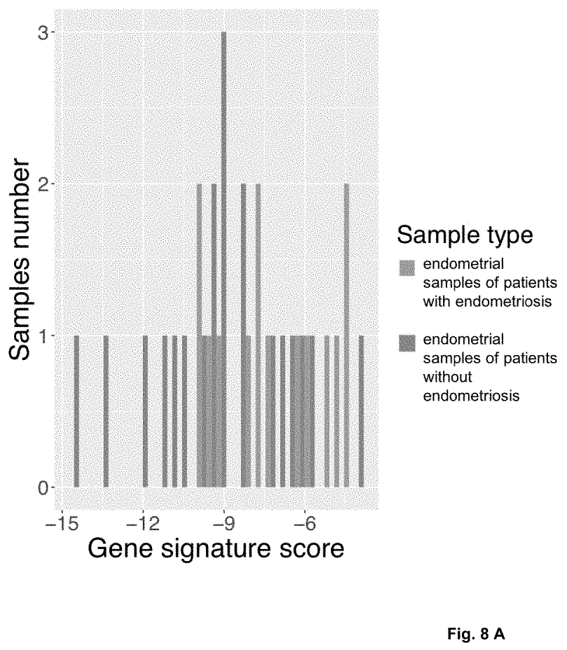

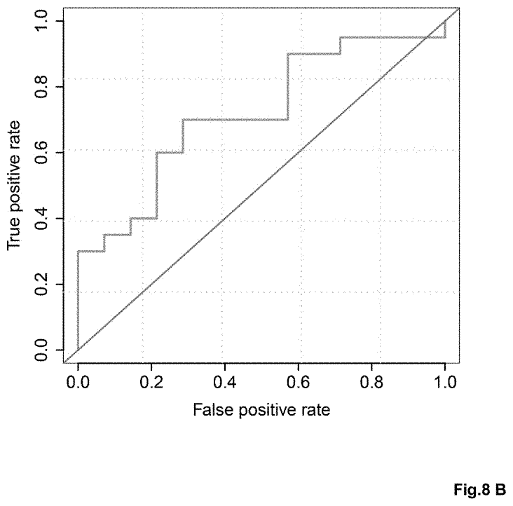

In order to independently validate the final prototype, we calculated 5-gene signature score on the external dataset GSE134056 (Akter S. et al. Machine Learning Classifiers for Endometriosis Using Transcriptomics and Methylomics Data//Front Genet. 2019;10:766). This dataset contains 38 RNA-seq profiles: 22 endometrial samples of women without endometriosis and 16 endometrial samples of women with endometriosis. However, none of the samples in the dataset passed pre-defined QC threshold of 3.5 million of uniquely mapped reads. We excluded two outliers according to clustering analysis and left only samples with at least 1 million of uniquely mapped reads to the HGNC genes to eliminate profiles of the lowest quality (FIG. 7). Thus, 14 endometrial samples of women with endometriosis and 20 endometrial samples of women without endometriosis were included into further analysis. It appeared that 5-gene signature score was capable of predicting the presence of endometriosis in this cohort of samples with AUC =0.72. (FIG. 8 A, B) However, the cut-off of 14 was not applicable in this case, possibly due to lower coverage, when compared to our original data.

Thus, the following approaches were applied in a complex manner for the first time during the analysis:

analysis of three groups of samples (endometrial samples and endometriosis lesions of patients with endometriosis; endometrial samples of women without gynecological diseases; samples of other tissues of controls without gynecological diseases - endocervix, ovarian epithelium);

application of the 5-fold cross-validation method to analyze differential gene expression, to validate preliminary gene signatures and to create a stable final gene expression signature for the diagnosis of endometriosis based on real clinical data.

The complex application of these approaches in combination with other methods of transcriptomic analysis (including the use of RNA sequencing, which makes it possible to obtain absolute quantification of various transcripts in a sample, rather than relative quantitative data, as is the case with the use of microarray technique), made it possible to identify the gene expression signature of endometrium that is characteristic for patients with endometriosis and allows to effectively differentiate endometrial samples of patients with endometriosis from endometrial samples of women without this disease.

Thus, this study used an RNA-seq gene expression analysis of endometrial samples and endometriotic lesions of patients with endometriosis in comparison with tissue samples of women without endometriosis in order to identify differentially expressed genes and to generate a gene signature capable of robust and precise diagnosis of endometriosis. Based on DESeq2 differential gene analysis, we devised machine learning procedures, including identification of DEGs specific to both endometrial samples and to endometriotic lesions versus tissue samples of women without endometriosis. The machine learning procedure included two steps. First, DEGs were extracted from a comparison between endometrial samples of patients with and without endometriosis. Second, genes were selected to provide a preliminary gene signature with a maximum AUC score for separation between endometriotic samples of patients with endometriosis and endometrial samples of patients without endometriosis. This two-step procedure returns relatively low numbers of DEGs (5-18 genes) and the subsequent five-fold cross validation procedure shows that the preliminary gene signatures generated in this way have high predictive power (AUC scores 0.95-1), which meets current standards of molecular diagnostics (Hajian-Tilaki K. Receiver Operating Characteristic (ROC) Curve Analysis for Medical Diagnostic; Test Evaluation//Caspian J intern Med. 2013;4(2):627-635). Moreover, the generated gene signatures shared a high number of common genes (5 genes) and enabled construction of a final gene signature. The final signature validation using the total dataset of 52 samples of patients with endometriosis (including endometrial samples and endometriotic lesions) and 14 tissue samples of both living and cadaveric patients without endometriosis showed that the final gene signature had a high AUC score (0.82) and high values of sensitivity (0.823) and specificity (1.0).

Our data strongly suggest that RNA-sequencing of endometrial samples and calculation of gene signature score for the genes LMNA, KDM6B, CIC, PER1, PPDPF could provide a measure of endometrial pathologic state seen in endometriosis. These genes were not found to be differentially expressed in the previous molecular studies of endometriosis, likely because all the genes of the final signature are downregulated in endometrial and endometriotic samples of patients with endometriosis compared with endometrial samples of women without endometriosis, whereas the previous studies were mainly concentrated on upregulated DEGs. Though KDM6B was not identified as a DEG in endometriosis, other related histone demethylases (KDM5 and KDM4C) were found upregulated in setting of severe endometriosis compared with the moderate stage of the disease (May K.E. et al. Endometrial alterations in endometriosis: A systematic review of putative biomarkers//Hum Reprod Update. 2011;17:637-653). Interestingly, the histone demethylases KDM6B and KDM5 play opposite roles in activation and regression of gene expression (Klein B. J. et al. The histone-H3K4-specific demethylase KDM5B binds to its substrate and product through distinct PHD fingers//Cell Rep. 2014;6(2):325-335; Jones S.E. et al.. Structural Basis of Histone Demethylase KDM6B Histone 3 Lysine 27 Specificity//Biochemistry. 2018;57(5):585-592), and here we demonstrate that KDM6B expression is downregulated in endometriosis, which is opposite to the previously reported change of KDM5 expression (upregulated in severe endometriosis compared with moderate disease) (May K.E. et al. Endometrial alterations in endometriosis: A systematic review of putative biomarkers//Hum Reprod Update. 2011;17:637-653). These findings emphasize an importance of histone demethylases-mediated epigenetic reprogramming in the pathogenesis of endometriosis. Additionally, siRNA-mediated downregulation of LMNA in human endometrial stromal cells was associated with a nuclear envelope assembly defect (Mortlock S. et al. Genetic regulation of methylation in human endometrium and blood and gene targets for reproductive diseases//Clin Epigenetics. 2019;11(1):49) that could lead to further perturbations of epigenetic gene regulation (Shevelyov Y. Y. et al. Role of Nuclear Lamina in Gene Repression and Maintenance of Chromosome Architecture in the Nucleus//Biochemistry (Mosc). 2018;83(4):359-369), and the onset of endometriosis. Moreover, the PPDPF gene was found differentially methylated between the proliferative and secretory phase in healthy endometrium during normal menstrual cycle (Mortlock S. et al. Genetic regulation of methylation in human endometrium and blood and gene targets for reproductive diseases//Clin Epigenetics. 2019;11(1):49). Therefore, although final signature genes were not determined to be differential in the previous studies of endometriosis, these genes could be tightly connected with the maintenance of a healthy endometrium state and normal menstrual cycle.

The proposed two-step framework of the NGS-sequencing data driven approach could be used in further research of complex gene expression biomarkers at the levels of individual genes or molecular pathways that determine the onset and progression of endometriosis (Buzdin A et al. Molecular pathway activation—New type of biomarkers for tumor morphology and personalized selection of target drugs//Semin Cancer BioL 2018;53:110-124). Moreover, the final 5-gene signature supports implementation of non-surgical diagnostics of endometriosis via endometrial sampling. Here, we propose a threshold value —14 for the gene signature score. This threshold value can be adjusted according to precise levels of type 1 and type 2 errors defined by clinicians. Alternatively, expression microarray, RT-qPCR, or proteomic assays based on the same characteristic genes can be developed for the diagnostics of endometriosis.

Despite the fact that to date numerous studies devoted to the investigation of genes and gene networks involved in the pathogenesis of endometriosis have been performed, where researchers analyze differentially expressed genes in endometrial samples and endometriotic lesions using microarray platforms and different sequencing techniques, no specific diagnostic tools have been created for the minimally invasive diagnosis of endometriosis.

Modern biomedical research often focused on development of a gene expression signature—a set of genes that reflects all specific molecular changes underlying a particular disease. We believe that such a signature can be used as a more effective diagnostic tool than any biomarker alone for such a multifactorial disease as endometriosis.

In this study we performed two-step differential gene analysis of endometrial samples and endometriotic lesions of patients with endometriosis in comparison with endometrial samples of women without endometriosis. Based on this analysis, we generated a characteristic signature of five genes downregulated in endometrial samples and endometriotic lesions of patients with endometriosis in comparison with endometrial samples of women without endometriosis (LMNA, KDM6B, CIC, PER1, PPDPF). Additional validation of final gene expression signature was done using other tissue samples of cadaveric patients without endometriosis and any other potentially genetically determined gynecological pathology (endocervix, ovarian surface epithelium). The method of five-fold cross validation of the proposed two-step procedure confirmed the ability to generate conservative and robust gene signatures having significant predictive power (AUC>0.85) using real-world clinical data. The marker gene set identified in our study could be used as the base for a non-surgical test system for molecular diagnosis of endometriosis.

An Example of Endometriosis Diagnosis using Identified Biomarkers

A 34-year-old female patient complaining of pelvic pain, aggravated by menstruation; dyspareunia, and infertility for 2 years. The patient underwent an endometrial aspiration biopsy, and the obtained endometrial sample was placed in a test tube with RNA-Later solution, which ensures the stabilization of RNA in cells, and sent to the laboratory for subsequent genetic analysis.

Analysis of LMNA, KDM6B, CIC, PER1, and PPDPF genes' expression was performed by next-generation RNA sequencing using the Illumina HiSeq 3000 according to the protocol recommended by the manufacturer as previously described in detail in the “Tissue-based procedures” section.

The analysis of the expression level of genes included in the gene expression signature of the endometrium in endometriosis demonstrated a downregulated expression of the LMNA, KDM6B, and CIC genes, which indicates a high probability of endometriosis presence in this patient.

Quantitative analysis of the gene expression signature in this patient revealed the following.

The normalized read counts of 5 genes were:
LMNA=351.6137, CIC=393.8073, PER1=355.4494, PPDPF=21.73612, KDM6B=409.1504.
   Calculation by formula

Gene signature score=−log₁₀(LMNA expression)−log₁₀(KDM6B expression)−log₁₀(CIC expression)−log₁₀(PER1 expression)−log₁₀(PPDPF expression)

showed the resulting value «-11.64».

According to conducted studies (see the section “Validation of final gene signature”), “−14” is the threshold for a gene expression signature, at which the ratio of errors of the first and second kind is minimal.

Since the “−11.64” value is more than the “−14” cut-off value, the assessment of the gene expression signature based on quantitative calculations also indicates a high probability of endometriosis presence in this patient.

The patient underwent diagnostic laparoscopy, where endometriosis lesions located on the pelvic peritoneum and uterosacral ligaments were detected and excised. The excised endometriosis lesions were sent for histologic examination, which confirmed the presence of endometriosis in this patient.

Thus, as a result of the conducted studies, gene expression signature of the endometrium of patients with endometriosis was identified which included the LMNA, KDM6B, CIC, PER1, and PPDPF genes, and which allows to effectively differentiate of endometrial samples of patients with endometriosis from endometrial samples of women without this disease. The analysis showed that the developed method has high sensitivity and specificity in the diagnosis of endometriosis.

Based on the data obtained, a non-invasive method for endometriosis diagnosis was developed based on the analysis of the identified genetic biomarkers included in the gene expression signature of the endometrium, and a system was developed for endometriosis diagnosis in a patient, containing hardware logic designed or configured to perform operations related to the analysis of the identified genetic biomarkers included in the gene expression signature of the endometrium samples of patients with endometriosis.

Notwithstanding the invention is described with reference to disclosed embodiments, it should be apparent to the experts in this field that detailed experiments are given only for the purposes of this invention illustration, and they shall not be considered as somehow limiting the scope of the invention. It should be apparent, that various modifications are possible without a departure from the essence of this invention.

We claim:

1. A method for endometriosis diagnosis, which includes analysis of the gene expression level in an endometrial sample and/or endometrial cells of the patient, and where a downregulated expression of at least two genes out of the following ones—LMNA, KDM6B, CIC, PERI and PPDPF—indicates the endometriosis presence in a particular patient.

2. The method according to claim 1, wherein the downregulated expression level of at least three genes or at least four genes out of the following ones—LMNA, KDM6B, CIC, PER1, and PPDPF—indicates the endometriosis presence in a particular patient.

3. The method according to claim 1, wherein the downregulated expression level of each of the following genes—LMNA, KDM6B, CIC, PER1, and PPDPF—indicates the endometriosis presence in a particular patient.

4. The method according to claim 1, wherein the endometrial sample and/or endometrial cells is obtained by endometrial biopsy, via isolation of endometrial cells from a menstrual blood sample, or via isolation of endometrial cells from a peripheral blood sample.

5. The method according to claim 6 wherein the endometrial biopsy means a brush biopsy or an aspiration biopsy.

6. The method according to claim 1, wherein the level of expression of the LMNA, KDM6B, CIC, PER1, and PPDPF genes in the patient's endometrial sample is determined by assessing the level of the corresponding mRNAs.

7. The method according to claim 6, wherein the mRNA analysis is performed by total RNA sequencing (NGS), using rtPCR, microarray hybridization, or custom panels designed to measure the gene expression.

8. The method according to claim 1, wherein the level of expression of the LMNA, KDM6B, CIC, PER1, and PPDPF genes in the patient endometrial sample is determined by quantification of the corresponding proteins.

9. The method according to claim 8, in which the quantification of the corresponding proteins is performed using mass spectrometry, or using ELISA, or using immunohistochemistry, or using protein microarrays, or using electrochemiluminescence.

10. The method according to claim 1, wherein the downregulated expression of any of LMNA, KDM6B, CIC, PER1, and PPDPF genes means an expression level lower than the corresponding one in an endometrial sample of a patient without endometriosis.

11. A method of endometriosis diagnosis, which includes gene expression analysis in an endometrial tissue sample and/or endometrial cells of the patient, which includes the following steps:
(a) defining a threshold for a specific device that measures the expression level of the LMNA, KDM6B, CIC, PER1, and PPDPF genes, calculating and minimizing the Type I error rate and Type II error rate of an endometriosis diagnosis in patients with known endometriosis status;
(b) measurement of the expression levels of the LMNA, KDM6B, CIC, PER1, and PPDPF genes in an endometrial tissue sample and/or endometrial cells of the patient;
(c) comparison of the values of the combination of gene expression levels measured in step
(d) with the threshold value defined for this device;
(e) when the gene signature value is above the threshold, the patient is diagnosed with endometriosis.

12. The method according to claim 11, wherein gene expression levels are measured using a device for NGS total RNA sequencing and the combination of LM NA, KDM6B, CIC, PER1, and PPDPF gene expression levels is calculated as the sum of the logarithms of normalized gene expression using the formula:
log₁₀(LMNA expression)−log₁₀(KDM6B expression)−log₁₀(CIC expression)−log₁₀(PER1 expression)−log₁₀(PPDPF expression).

13. The method according to claim 12, wherein the NGS device for total RNA sequencing is an illumina HiSeq-3000 and the threshold is “−14”.

14. A system for endometriosis diagnosis in a patient, comprising hardware logic designed or configured to perform operations including:
(a) receiving information regarding expression levels of the LM NA, KDM6B, CIC, PER1, and PPDPF genes in an endometrial tissue sample and/or endometrial cells taken from the patient,
(b) applying gene expression levels to a predictive model relating expression levels of mentioned genes to endometriosis; and
(c) evaluating of an output of said predictive model to assess the probability of endometriosis presence in a patient,
while the presence of endometriosis in the patient is indicated by the downregulated expression of at least two genes out of the following five genes—LMNA, KDM6B, CIC, PER1, and PPDPF.

15. The system according to claim 14, wherein the endometriosis presence in the patient is indicated by the downregulated expression of at least three genes or at least four genes out of the following five genes—LMNA, KDM6B, CIC, PER1, and PPDPF.

16. The system according to claim 14, wherein the endometriosis presence in the patient is indicated by the downregulated expression of each of the following genes—LMNA, KDM6B, CIC, PER1, and PPDPF.

17. The system according to claim 14, wherein the system comprises a microarray.

18. The system according to claim 14, wherein the system comprises a sequencer.

19. The system according to claim 14, wherein the system comprises an RT-PCR system (reverse transcription-quantitative real-time polymerase chain reaction (RT-PCR) system), a mass spectrometer, a luminometer, a spectrophotometer, or a fluorometer.
